(19) United States
(12) Patent Application Publication
(10) Pub. No.: US 2020/0242133 A1
(43) Pub. Date: Jul. 30, 2020

(54) REDUCING A SEARCH SPACE FOR A MATCH TO A QUERY

(71) Applicant: Babylon Partners Limited, London (GB)

(72) Inventors: Georgios STOILOS, London (GB); Szymon WARTAK, London (GB); Damir JURIC, London (GB); Jonathan MOORE, London (GB); Mohammad KHODADADI, London (GB)

(21) Appl. No.: 16/262,054

(22) Filed: Jan. 30, 2019

(51) Int. Cl. G06F 16/28; G06F 16/242; G16H 50/20

(52) U.S. Cl. CPC G06F 16/285; G06F 17/2785; G16H 50/20; G06F 16/243

(57) ABSTRACT

Methods for reducing the number of potential matches of entries in a database to a user inputted query are provided. In one aspect, a method includes receiving a user inputted query, identifying a plurality of candidate entries in said database that provide a match to said user inputted query, and grouping the plurality of candidate entries on the basis of their associated semantic type. The method also includes selecting the group with the largest number of entries, and transmitting a request to a user to select between the entries in the group with the largest number of entries. Systems and machine-readable media are also provided.

### FIELD

Embodiments described herein relate to methods and systems for finding a match to query in a database, for example, dialogue systems, question and answering system, or decision support system.

### BACKGROUND

Much attention from both academia as well as industry has gathered around task-oriented dialogue systems (TODS) and chatbots in the last decade. Many such systems are built for entertainment or business purposes while others with the goal of reducing the cost, increasing the speed, or improving the quality of services delivered to end-users. Examples of domains for which such services are offered include, banking and financial services, online shopping, intelligent device control, and more.

A field that has drawn considerable attention to such technologies is healthcare and symptom-checking. The lack of resources to handle the ever-increasing demand for better healthcare, and the need for chronic disease management make such solutions appealing as they promise immediate response and assessment reducing the need to visit emergency rooms and local practices. Consequently, symptom-checkers and health assessment dialogue systems have been developed. In such a scenario a user inputs a text like “I have a fever” and the relevant nodes (symptoms/evidence) in some statistical-based inference model need to be activated in order to initiate the symptom-checking process.

Previous approaches to dialogue systems assume that users express their intention in a precise and clear way like “I have a stomach ache,” “I want to book a flight,” and “I want a loan” from which the relevant terms can be extracted using ML or rule-based techniques. However, especially in the medical domain, this is usually not the case making it hard to accurately match user input to nodes of the model. First, user input may be highly vague like “I have a pain” in which case several nodes in the statistical inference engine may be relevant like “Abdominal Pain,” “Low Back Pain,” and many more. Second, users may actually be experiencing something slightly different from what they report. For example, in the previous case, the user may actually have had an injury which results is his/her pain. Third, user text may be highly colloquial like “I feel my head will explode” or “My heart is running like hell,” and more. In all these cases, it is impossible to match user input to the right nodes in the inference model. Fourth, there is usually a gap between the formally medical language used to encode the nodes of the engine with the terms used by users. For example, such an engine may contain a node like “Periumbilical pain” and “sputum in throat,” however, users will not use such formal language to report their symptoms.

The above issues are partially solved using similarity-based retrieval techniques like embeddings, which can retrieve the top-k most “similar” symptoms (or entities from a KB in general) to the user input. Then, users would need to review the list and select the most appropriate one. Although this approach does improve the recall of dialogue systems, it suffers from several drawbacks. First, it is clearly not user friendly and users may still find it difficult without any “professional” help to browse through the list, understand the differences of the (possibly similar in many cases) symptoms, and select the right one. Second, there is clearly a limit to the number of items in the list of symptoms that users can browse through to find the desired option. The size of this list is even smaller in speech-based dialogue systems where the list needs to be read to them.

### BRIEF DESCRIPTION OF THE FIGURES

The drawings are as follows:

FIG. 1 is a schematic of a system in accordance with an embodiment;

FIG. 2 is a chart showing dialogue flow controlled by a method in accordance with an embodiment;

FIG. 3 is a flowchart depicting an overview of the steps of a method in accordance with an embodiment;

FIG. 4 is a chart showing dialogue flow controlled by a method in accordance with an embodiment;

FIG. 5 is a flowchart showing an overview of the generation of candidates for the best match;

FIG. 6 is a detailed flow chart showing the generation of candidates for the best match;

FIG. 7 is a flow chart of a candidate selection process in accordance with an embodiment;

FIG. 8 is a schematic of a question generation process in accordance with an embodiment;

FIG. 9 is a flow chart showing a database pre-processing method in accordance with an embodiment;

FIG. 10 is a schematic of a system in accordance with an embodiment.

### DETAILED DESCRIPTION

In an embodiment, a method for reducing the number of potential matches of entries in a database to a user inputted query is provided, the method comprising: receiving a user inputted query; identifying a plurality of candidate entries in said database that provide a match to said user inputted query; grouping the plurality of candidate entries on the basis of their associated semantic type; selecting the group with the largest number of entries; and transmitting a request to a user to select between the entries in the group with the largest number of entries.

The disclosed system addresses a technical problem tied to computer technology and arising in the realm of computer networks, namely the technical problem of providing an efficient method of determining the best match to an entry in a database to a query where there are many possible matches. This is achieved by sending a further query to the user; the further query sent to the user is designed to allow the number of possible matches to be narrowed down in an efficient manner. The disclosed system solves this technical problem with a technical solution, namely by identifying possible entries to which the query relates and groups these topics by semantic type. A request is then transmitted to the user to select from options based on the group selected with the largest number of entries. This allows the search space to be quickly reduced and therefore results in a reduction of network traffic since the number of queries required to produce the eventual response is reduced.

For example, if the system relates to a medical system and a user inputs “I have a rash,” the topics identified might be, for example, “bumpy rash,” “small pimples,” “face rash,” “body rash,” “arm rash,” etc. In this simplified example, there are two groups by semantic type: Group 1—“bumpy rash,” “small pimples,” and Group 2 “face rash,” “body rash,” and “arm rash.” For Group 1m, the semantic type relates to the appearance of the rash and for group 2, the semantic type defines the location of the rash. In this simplified example, the user may then be presented with a response “Where is the rash: 1) face; 2) body; or 3) arm?”

The best match to a query is required in many different types of situations; for example, in a medical diagnosis system, it is important to correctly identify the symptoms of the user. Such systems may comprise a probabilistic graphical model (PGM) that describes the probabilistic relationship between symptoms and possibly causes. To use such a system, it is necessary to identify (or “activate”) the node that represents the best match to the inputted query. This is a different problem to just selecting multiple possible matches.

In one embodiment, the entries in the database are concepts in a knowledge base and are stored in the form of triples, said triples comprising a first concept, a second concept, and a relation between the first concept and the second concept, wherein the relation is selected from a plurality of relations, one of which is semantic type. Examples of the possible semantic types are: body part, observable entity, abnormal body part, substance, organism, qualifier value, clinical finding, anatomy qualifier, spatial qualifier and time patterns, and time duration.

The transmitted request may additionally comprise information from matches other than those in the selected group. For example, if the semantic type with the largest group is location, and the user reports that they have a pain, the request to the user might comprise a question such as “Where is the pain?—head, arm . . . etc” and possibly a further question—“Is the pain sharp?” in addition to the selection.

In an embodiment, the user is asked to select between the group with the largest number of entries if the largest number of entries is in excess of a threshold.

The initial candidates can be determined using a number of methods.

In an embodiment, identifying a plurality of candidates comprises determining the nearest neighbors from said database entries when mapped to the same embedded space as the query.

In an embodiment, the entries in the database are concepts in a knowledge base and are stored in the form of triples, said triples comprising a first concept, a second concept, and a relation between the first concept and the second concept, wherein the relation is selected from a plurality of relations, one of which is semantic type, further, a subset of the concepts in the knowledge base are target concepts, wherein said method is adapted to provide matches to said target concepts, wherein said matches to target concepts are determined by: annotating the query by selecting concepts from the knowledge base that have a label that is similar to the query; determining matches to target concepts from the selected concepts by determining from the knowledge base all concepts descended from the selected concepts and keeping only those that are also target concepts.

In an embodiment, the entries in the database are concepts in a knowledge base and are stored in the form of triples, said triples comprising a first concept, a second concept, and a relation between the first concept and the second concept, wherein the relation is selected from a plurality of relations, one of which is semantic type, further, a subset of the concepts in the knowledge base are target concepts, wherein said method is adapted to provide matches to said target concepts, wherein said matches to target concepts are determined by: annotating the query by selecting concepts from the knowledge base that have a label that is similar to the query to obtain first selected concepts; identifying the semantic types of these first selected concepts; annotating the query by selecting concepts from the target concepts that have a label that is similar to the query to obtain second selected concepts; identifying the semantic types of these second selected concepts; and determining matches to said target concepts from second selected concepts that have a semantic type that matches with one of the semantic types of the first selected concepts.

In the above embodiment, matches to said target concepts are determined from second selected concepts that have a semantic type (or are linked to concepts in the knowledge base with a semantic type that matches with one of the semantic types of the first selected concepts).

In a further embodiment, determining matches to target concepts comprises a primary method followed by a reserve method. In the primary method, the query is annotated by selecting concepts from the knowledge base that have a label that is similar to the query, then matches to target concepts are determined from the selected concepts by determining from the knowledge base all concepts descended from the selected concepts and keeping only those that are also target concepts.

In the event that the primary method does not yield results, a reserve method is used where annotating the query is performed by: selecting concepts from the knowledge base that have a label that is similar to the query to obtain first selected concepts; identifying the semantic types of these first selected concepts; annotating the query by selecting concepts from the target concepts that have a label that is similar to the query to obtain second selected concepts; identifying the semantic types of these second selected concepts; and determining matches to said target concepts from second selected concepts that have a semantic type that matches with one of the semantic types of the first selected concepts.

In a further embodiment, the method comprises pre-processing the database prior to identifying a plurality of candidate entries, wherein the pre-processing comprises producing a triple for indirectly related concepts which are related through multiple directly related concepts.

In a further embodiment, further pre-processing of the database prior to identifying a plurality of candidate entries, each concept in the database having a label, the method of pre-processing comprising: identifying secondary concepts from the label; determining a relationship from the label between a secondary concept identified in the label and the concept; and saving the concept, secondary concept, and relationship as a triple.

The embodiments described herein can be used to process textual data. Text can be used to convey complex meaning and knowledge and this can be done in various different but equivalent ways. However, the techniques for extracting the meaning conveyed in text and reasoning with it are still not well developed, as text understanding and reasoning is a highly difficult problem.

Knowledge Bases (KBs) have started to play a key role in many academic and industrial-strength applications like recommendation systems, dialogue systems, and more. In such applications, users form their requests using short queries, e.g., “I want to book a flight,” “I am looking for Italian Restaurants,” “I have a fever,” and so forth, and these should be used to activate the proper KB entities which are used to encode or control the background application logic. In particular, symptom-checking dialogue-systems (SCDSs) have attracted considerable attention due to their promise of low-cost and continuous availability and many academic and industrial systems are also starting to emerge.

All previous approaches assume that users express their requests in a precise and clear way like “I have a stomach ache,” “I want to book a flight,” and “I want a loan” from which the relevant terms can be extracted using ML or rule based techniques and mapped to proper KB entities. However, this assumption leads to less natural human-computer interaction and is bound to fail in complex applications like symptom-checking. For example, in such a scenario, user input may often be highly vague like “I have a pain” in which case several entities in the KB may be relevant like “Abdominal Pain,” “Low Back Pain,” and many more or highly colloquial like “I feel my head will explode.” In all these cases, it is impossible to match user input to the right entities in the inference model. In addition, there is usually a gap between the formal medical language encountered in medical KBs and the terms used by users. For example, a symptom-checker may contain a node like “Periumbilical pain” and “sputum in throat,” however, users will never use such formal language to report their symptoms.

The above issue is partially solved by using similarity-based retrieval techniques like embeddings which can retrieve the top-k most “similar” KB entities and then ask the user to select from them. However, this approach suffers from several drawbacks. First, it is clearly not user friendly and, second, especially in the symptom-checking scenario, users may still find it difficult to browse through the list and understand the differences of the (possibly similar in many cases) symptoms. Third, there is clearly a limit to the number of candidate entities that users can browse over which drops even more for speech based dialogue systems where the list needs to be read out.

The embodiments described herein address the above issues and provide a framework and algorithm that can be used to “guide” users into associating to their initial query some entity from a pre-defined set of target entities that most closely matches their intention. First, an initial “small” subset of the target entities is extracted using the hierarchy of the KB together with statistical techniques like embeddings. Second, the properties of these candidates in the KB are used to group them into categories. These categories are then used to ask the user specific questions. For instance, in an example, the system could ask the user “In which area of your body is your pain?” with potential answers “In eye,” “On Leg,” etc. The effectiveness of the grouping algorithm depends on the number of properties that the target entities share. Further embodiments also relate to an entity enrichment step that uses information extraction techniques and a custom scoring model to prioritise the verification of the newly extracted properties.

The embodiments described herein do not assume a fixed set of frames with slots that need to be filled and pre-defined questions that can be used for these purposes. In contrast, the target set may contain highly diverse entities and the user query may match any subset of them. Hence, the algorithm is highly flexible and dynamic and is able to handle highly diverse and broad domains like symptom-checking. Further, the approach is largely unsupervised, as it does not depend on any pre-existing corpora of sample dialogues or user queries from logs where a mapping from user text to KB entities can be learned. Compared to guided navigation and faceted search, the approach is implemented as a short dialogue that presents one question at a time and has to prioritise which question to ask first. In contrast, faceted navigation is prevalently click-based and all facets with result counts and current candidates are presented to the user.

Ontologies have been developed to capture and organize human knowledge and Semantic Web standards e.g., RDF and OWL is one set of tools for encoding such knowledge in a formal machine-understandable way. Ontologies can be used to describe the meaning of textual data and provide the vocabulary that is used by services to communicate and exchange information or by users to access and understand the underlying data.

SNOMED (Systematized Nomenclature of Medicine) is a systematically organised computer-processable collection of medical terms providing codes terms, synonyms, and definitions used in clinical documentation. SNOMED has four primary core components:

Concept Codes—numerical codes that identify clinical terms, primitive or defined, organized in hierarchies.

Descriptions—textual descriptions of Concept Codes.

Relationships—relationships between Concept Codes that have a related meaning.

Reference Sets—used to group Concepts or Descriptions into sets, including reference sets and cross-maps to other classifications and standards.

Other knowledge bases such as NCI, UMLS, and more have been used extensively in both academic and industrial applications.

Concepts in SNOMED are defined using codes, e.g., concept 161006, 310497006, etc. For readability reasons hereon, instead of using codes, labels will be used that represents the intended real-world meaning of a concept. For example, SNOMED concept 161006 intends to capture the notion of a “Thermal Injury” while concept 310497006 is the concept of a “Severe Depression.” Hence, instead of “concept 310497006”-“concept SevereDepression” will be used.

An ontology can only contain a finite number of “elementary” concepts (or atomic concepts) which are building blocks for other real-world notions which may or may not be pre-defined in the ontology. Hence, these elementary concepts can be used to build concepts that are not necessarily pre-defined in SNOMED or the like.

For example, concept “recent head injury” can be defined in (at least) the following two ways using SNOMED elementary concepts:
C₁:=RecentInjury Π ∃findingSite.HeadStructure
C₂:=HeadInjury Π ∃temporalContext.Recently

A definition of the terms used in this application is given in Appendix A.

In the above embodiment, the topics can be represented by concepts.

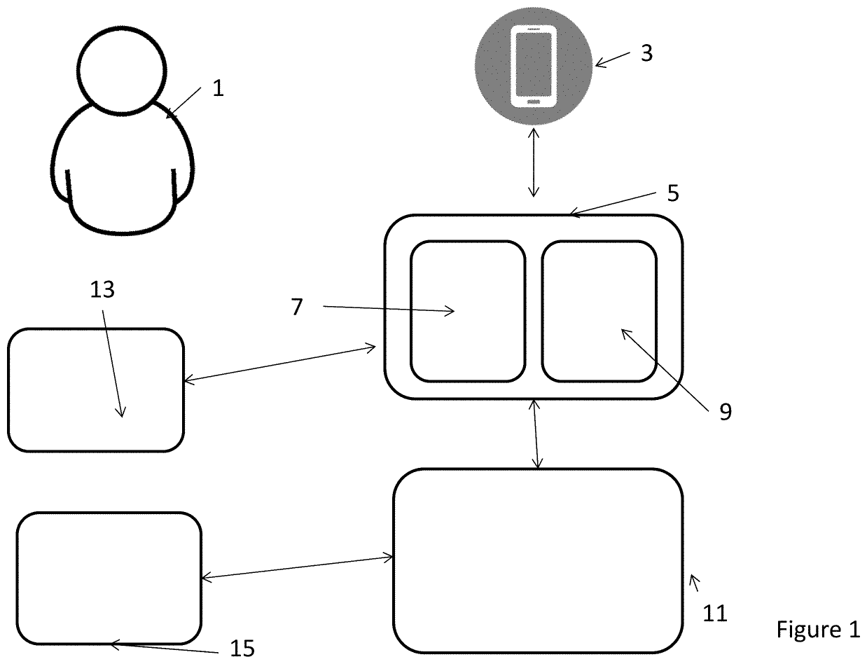

FIG. 1 is a schematic of a diagnostic system. In one embodiment, a user 1 communicates with the system via a mobile phone 3. However, any device could be used, which is capable of communicating information over a computer network, for example, a laptop, tablet computer, information point, fixed computer, etc. The user can input their query using speech or input text. Where speech is inputted, a speech recognition system is used.

The mobile phone 3 will communicate with interface 5. Interface 5 has two primary functions; the first function 7 is to take the words uttered by the user and turn them into a form that can be understood by the inference engine 11. The second function 9 is to take the output of the inference engine 11 and to send this back to the user's mobile phone 3.

In some embodiments, Natural Language Processing (NLP) is used in the interface 5. NLP helps computers interpret, understand, and then use every day human language and language patterns. It breaks both speech and text down into shorter components and interprets these more manageable blocks to understand what each individual component means and how it contributes to the overall meaning, linking the occurrence of medical terms to the Knowledge Base. Through NLP, it is possible to transcribe consultations, summarise clinical records, and chat with users in a more natural, human way.

However, simply understanding how users express their symptoms and risk factors is not enough to identify and provide reasons about the underlying set of diseases. For this, the inference engine 11 is used. The inference engine is a powerful set of machine learning systems, capable of reasoning on a space of >hundreds of billions of combinations of symptoms, diseases, and risk factors, per second, to suggest possible underlying conditions. The inference engine can provide reasoning efficiently, at scale, to bring healthcare to millions.

In an embodiment, the Knowledge Base 13 is a large structured medical knowledge base. It captures human knowledge on modern medicine encoded for machines. This is used to allow the above components to speak to each other. The Knowledge Base keeps track of the meaning behind medical terminology across different medical systems and different languages.

In an embodiment, the patient data is stored using a so-called user graph 15.

The systems and embodiments described herein are related to operations within the interface 5.

In an embodiment, a user input is received from the mobile phone 3 or other input device. One of the tasks of the interface 5 is to establish from the user input, the concepts to which the input relates from the knowledge base.

However, when a user inputs a query, it is likely that the user will express the query in vague terms (e.g., “I have a rash”) to link to just one concept in the knowledge base. Also, the user is likely to express themselves using colloquial language (e.g., “I feel awful”), which might not directly correspond to any concept in the knowledge base.

In an embodiment, one of the tasks of the user interface 5 is to identify a plurality of concepts of interest from a Knowledge Base K and a text query q. The next stage is to then provide a mechanism to guide the user to a single concept C∈TargetCons that expresses best the intention of q. Concepts in TargetCons can be symptoms in some symptom-checking application (e.g., Fever, Headache, Nausea) or concepts related to places in a holiday booking system (e.g., Beach, SkyResort, WarmPlace), and more. In an embodiment, the interface is for a medical diagnosis system as described with reference to FIG. 1. The nodes in the PGM are the target concepts (TargetCons). These TargetCons are also a subset of the concepts of the knowledge base.

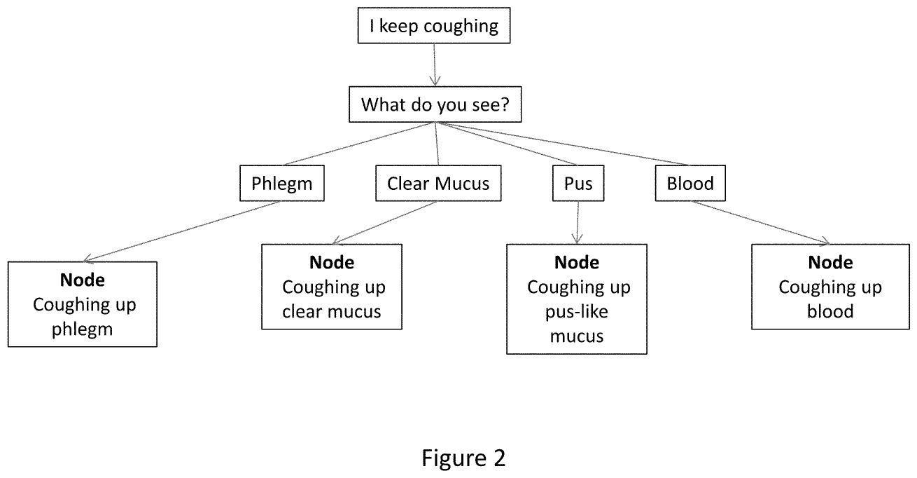

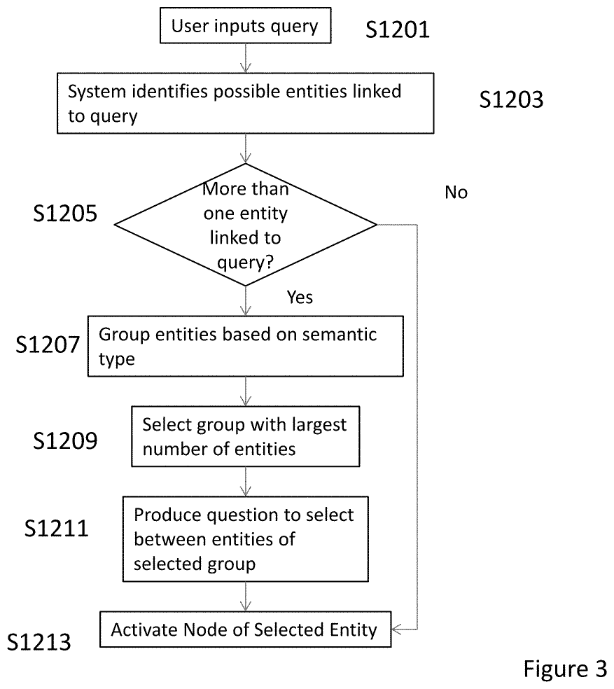

A high-level view of the system will now be described with reference to FIGS. 2 and 3. FIG. 2 is a schematic of a high-level dialogue flow and FIG. 3 is a simplified flow chart.

In FIG. 2, the user inputs a phrase in S1201 of FIG. 3, in FIG. 2; this is the phrase “I keep coughing.” In the system of FIG. 1, the interface 5 needs to be able to assign this query to the most appropriate node in the inference engine (PGM). Once a node in the PGM has been identified, the diagnosis process can start since a symptom has been identified and this symptom will be linked to multiple possible causes. How the next question is determined once a node has been identified corresponding to the user's question is outside the scope of this application. This application is concerned with identifying the most appropriate single concept that corresponds to a node in the PGM.

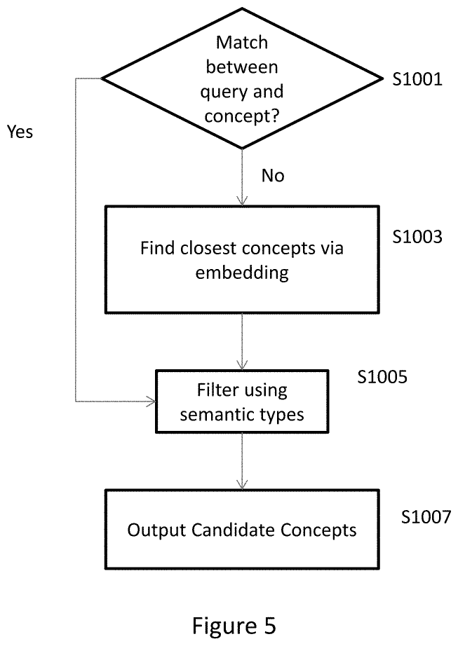

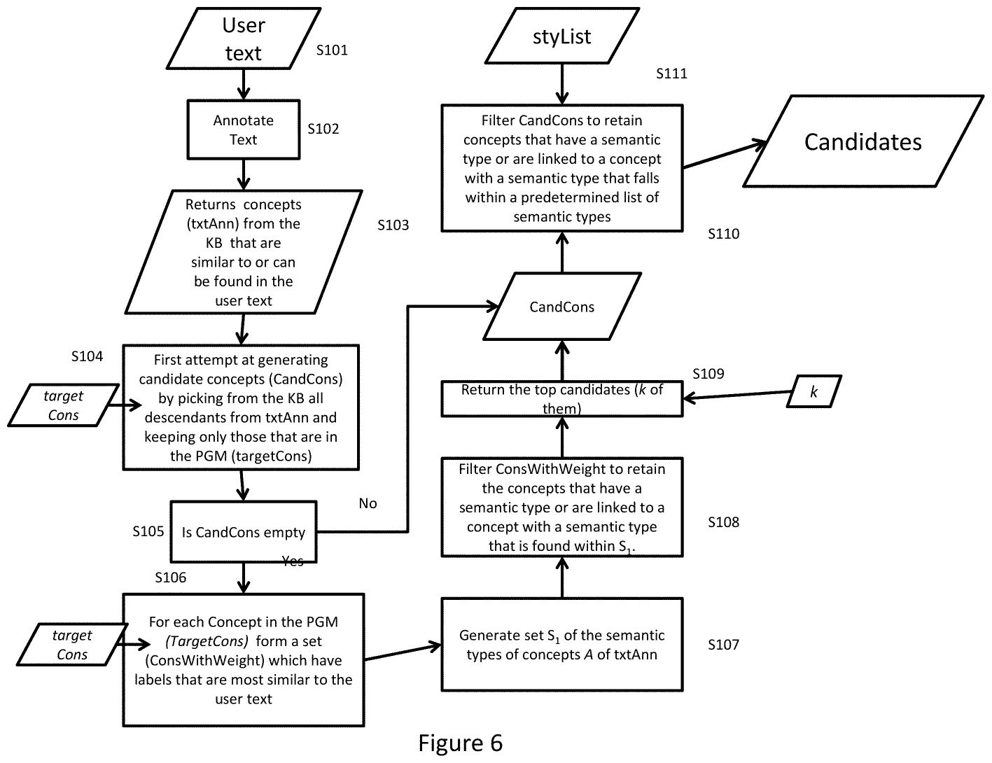

In step S1203, the system then identifies possible entities that are linked to the query. In an embodiment, these are concepts that correspond to nodes in the PGM. How this will be achieved will be explained with reference to FIGS. 5 and 6 below. In step S1205 it is determined that just one entity is identified in step S1203, and the node is activated in step S1213.

However, if the query inputted by the user was not precise, it is likely that more than one entity will be identified in step S1203 and therefore, step S1205 is likely to pass to step S1207. Here, the entities will be grouped on semantic type.

Referring back to the dialogue flow of FIG. 2, the query “I keep coughing” causes (among others) the following concepts to be identified in step S1203: coughing; coughing at night; dry cough; coughing up phlegm; coughing up clear mucus; coughing up pus like mucus; coughing up blood.

In step S1207, the entities are then grouped dependent on what will be termed their associated semantic type. This can be thought of as how the identified entities map to a broader entity. For example, “coughing up phlegm” can be expressed as coughing and phlegm that are linked (in layman's terms) by the relationship—“what is coughed up?” Out of the identified concepts above—coughing up phlegm; coughing up clear mucus; coughing up pus like mucus; coughing up blood all relate to “what is coughed up” whereas “coughing a night” refers to the time/frequency of the cough. It should be noted that in the above, the associated sematic types are the semantic types of the concepts to which the entities are linked via properties. Taking the above example, the grouping is performed w.r.t. the semantic type of “pleghm,” “mucus,” etc., which is BodySubstance, whereas the semantic type of the nodes themselves is ClinicalFinding.

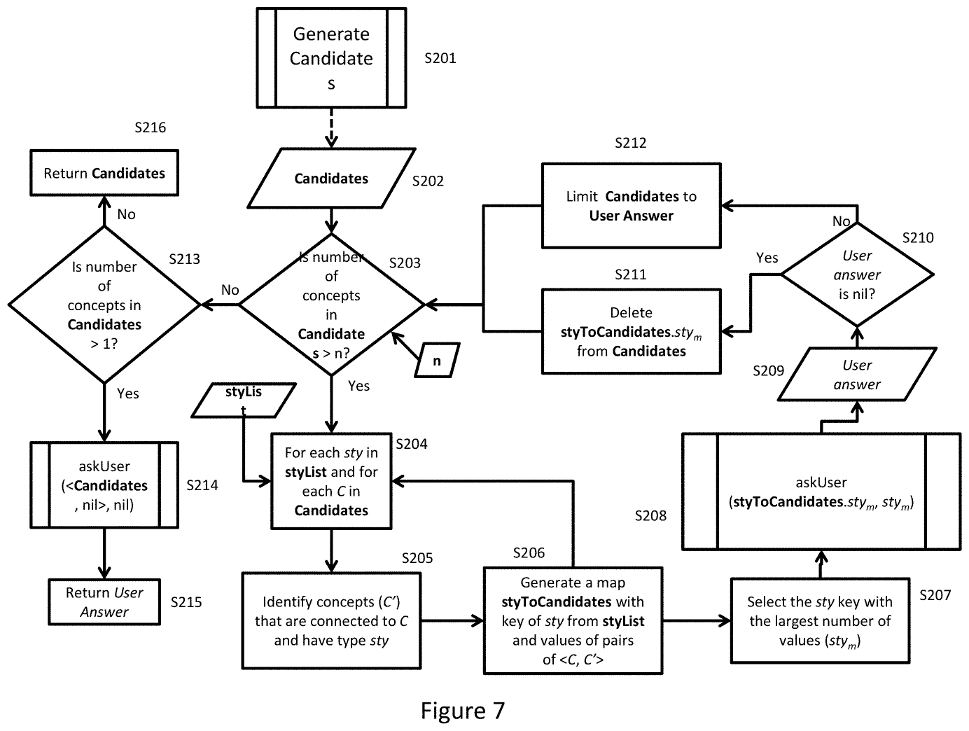

How this grouping is performed will be described with reference to FIG. 7. In step S1209, the group with the largest number of entities is selected as the basis for the next question in step S1211, which in FIG. 2 is shown as “What do you see?” and this gives the options as: phlegm; clear mucus; pus; and blood. The final four of these link directly to a single concept in the PGM. So, if the user selects one of these, then a single node in the PGM is selected in step S1213 and then further diagnosis can take place using the PGM and inference engine to drive questions.

In addition to the above selections shown, there will be a “none of the above option.”

To understand how the above dialogue is handled, some further basic nomenclature will be described.

For a set of tuples of the form tup={ [symbol] k₁ν₁ [symbol] , [symbol] k₂ν₂ [symbol] , . . . [symbol] kₙνₙ [symbol] } and for i∈{1,2}, πᵢtup is called the projection of tup on the first (resp. second) argument and returns the sets π₁={k₁, k₂, . . . , K_{N}} (resp. π₂={ν₁, ν₂, . . . , ν_{N}}).

A map is a collection of key/value pairs. In an embodiment, semi-structured maps can be allowed, that is, maps where the values of different keys may be of a different type. For Map, a map, and k, some key, the notation Map:k can be used to denote the value associated with k. If no value exists for some key k, then Map:k:=v means that a new key k is added to the map and its value is set to v.

### Considering now a Knowledge Base:

Let C and R be countably disjoint sets of concepts and properties. Concepts and properties are uniquely identified using IRIs. A Knowledge Base (KB) is a tuple [symbol] [symbol] , [symbol] , μ, ρ, δ [symbol] where [symbol] is a set of subject, property, object triples of the form <s p o> like in the RDF standard, [symbol] is a subset of concepts from [symbol] called semantic types (stys), μ is a mapping from every concept in [symbol] to a non-empty subset of [symbol] and both ρ and δ are mappings from each R∈R to a possibly empty subset of C. In addition, it is assumed that every concept C is associated with a preferred label and a user-friendly label. This can be specified using triples <C prefLabel “pref label”> and <C laymanLabel “layman label”>. For convenience, the notation, C. [symbol] and C.lay is used to refer to these two labels and the notation C.p is used to refer to the set {C′|(C p C′)∈ [symbol] }. Finally, for a concept C the function μ⁺(C) is used to denote the set μ(C)∪U_{C′∈C.p}μ(C′).

Intuitively, stys (semantic types) denote general/abstract categories of interest in the KB and are used to group other concepts while ρ and δ define the range and domains of properties. For example, in a medical KB there can be stys like Disease, Drug, BodyPart and the like, and then Malaria can have has sty Disease. This information can also be encoded within [symbol] using triples of the form <Disease is_sty true> and <Malaria has_sty Disease>. Moreover, for concept MyocardialInfarction:
MyocardialInfarction. [symbol] =“Myocardial infarction” and
MyocardialInfarction:lay=“Heart attack.”

A property that is used from the RDF standard is subClassOf (⊆ for short) that can be used to specify that the subject of a triple implies the object, e.g., <VivaxMalaria subClassOf Malaria>.

It is said that C is subsumed by D w.r.t. a KB [symbol] if [symbol] [symbol] [symbol] C subClassOf D [symbol] where [symbol] is entailment under the standard FO-semantics. For simplicity and without loss of generality, it is assumed that μ is closed under subClassOf in the following sense, if sty ∈μ(A) and [symbol] [symbol] [symbol] sty subClassOf sty′ [symbol] then sty′ ∈μ(A).

For efficient storage and querying, it can be assumed that the KB is loaded to a triple-store employed with (at least) RDFS forward-chaining reasoning. Forward-chaining implies that inferences under the RDFS-semantics are materialised during loading. Hence, if [symbol] [symbol] [symbol] C subClassOf D [symbol] under the RDFS-semantics, then [symbol] C subClassOf D [symbol] ∈ [symbol] .

In an example, a problem is studied where: given a subset of concepts TargetCons from a KB [symbol] (target concepts or concepts of interest) and a text query q provide mechanisms to guide the user to a single C E TargetCons that expresses best the intention of q. Concepts in TargetCons can be symptoms in some symptom-checking application (e.g., Fever; Headache; Nausea) or concepts related to places in a holiday booking system (e.g., Beach; SkyResort; WarmPlace), and more. The restriction of single concepts is important and is motivated by the fact that systems like virtual assistants and task-oriented dialogue systems require a single entity to be activated in order to proceed with their application logic.

Next, an example of how this can be performed in accordance with an embodiment will be described with reference to FIGS. 1 to 10.

The embodiments described herein are able to deal with vague or imprecise user inputted text.

In an example, a user intends to a use a symptom-checking dialogue system (SCDS) which can be a system of the type described with reference to FIG. 1. In such a scenario, a user can enter text like Q=“I have a rash.” Such a statement is quite vague and an SCDS is likely to contain more specific symptoms like CircularRash, RashInAbdomen, RashInArm, CircularRash, and BumpyRash, all of which are relevant to user's inputted text. Even when people go to see a doctor, the doctor usually needs to ask a series of questions about the nature of the reported symptom, like its location, its onset, severity, and more, in order to understand patient conditions better.

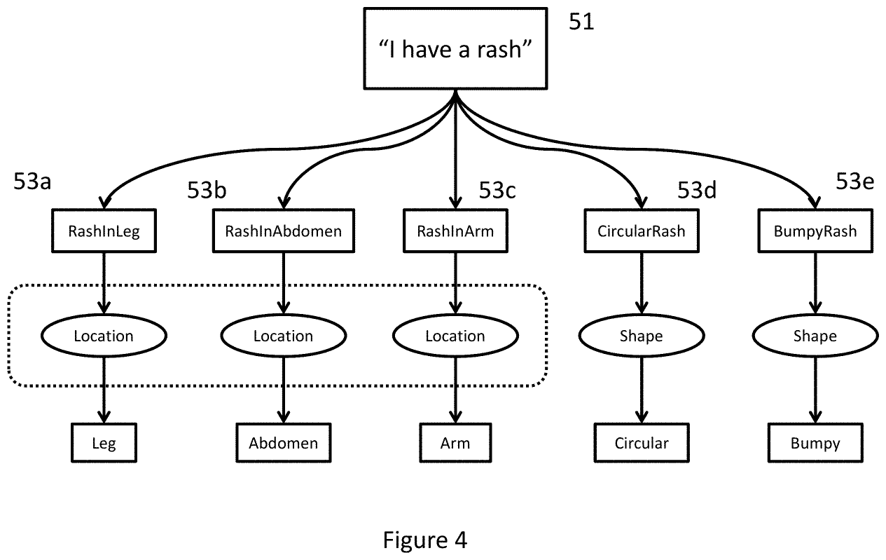

Such a scenario is shown in FIG. 4. Here, the user input “I have a rash” in 51. The five possible entities above CircularRash 53a, RashInAbdomen 53b, RashInArm 53c, CircularRash 53d, and BumpyRash 53e all correspond to entities in the PGM. However, the single query “I have a rash” does not allow one to be clearly identified.

A first challenge in the above scenario is to determine an initial and highly relevant set of concepts from the set of concepts that the dialogue system “understands” (TargetCons). Several different alternatives can be considered for this step. An approach which is actually used in some commercial SCDS is by using sentence embeddings (see Yang et al Universal Sentence Encoder CoRR (2018)).

In an embodiment, all labels of the symptoms in TargetCons are embedded to produce a vector for each label in an embedded space. The user input is then embedded into the same space with the entities in TargetCons and the top-k closest vectors corresponding to labels in the knowledge base can be returned. For the above two operations, two functions vectorize and sim are assumed. The former takes as input some text [symbol] and returns a vector in some vector space while the latter is the angular distance between two vectors.

As a further example, the same input sentence Q and the concepts of the SCDS mentioned above are again used. However, here a text annotator is trained on a medical KB [symbol] like SNOMED CT. When applied on Q, the annotator will return concept C:=Rash. It is expected that in a medical ontology like SNOMED CT, concept C is somehow semantically related to the symptoms in TargetCons that are potentially relevant to the patient condition. For example, RashInAbdomen is expected to be a sub-concept of C.

Relevance between two concepts can be defined in a strong way as “all those D∈TargetCons such that K [symbol] D [symbol] C” or maybe also in a more loose way as “all D E TargetCons such that some path of triples from D to C exists in [symbol] .”

In an embodiment, both the embedding method of looking for the closest candidates and the method of generating candidates from a pathway of triples between the query and the target concepts above are used to develop method Generate Candidates in accordance with an embodiment.

The method can be implemented using the below Algorithm 1 and will be explained with reference to FIGS. 5 and 6:

Algorithm 1 GenerateCandidates [symbol] (txt, TargetCons, k, styList)

| | |
|---|---|
| 1: | txtAnn := AnnotateText [symbol] (txt) |
| 2: | CandCons := {C \| [symbol] C [symbol] A [symbol] ∈ [symbol] , A ∈ txtAnn, C ∈ TargetCons} |
| 3: | if Cand Cons = = ∅ then |

| | |
|---|---|
| 4: | ConsWithWeight := { [symbol] C, sim(vectorize(C, [symbol] ), vectorize(txt)) [symbol] \| C ∈ TargerCons} |
| 5: | S₁ := ∪_{A∈txtAnn} μ(A) |
| 6: | ConsWithWeight := { [symbol] C, n [symbol] ∈ ConsWithWeight \| S₁ ∩ μ⁺ (C) ≠ ∅} |
| 7: | Cand Cons := π₁top(k, ConsWithWeight) |

| | |
|---|---|
| 8: | end if |
| 9: | CandCons := {C ∈ CandCons \| μ⁺ (C) ∩ styList ≠ ∅} |
| 10: | return CandCons |

The method takes as input some text, as a set of concepts of interest (these can be symptoms of some SCDS but any other set of target concepts can be used), a positive integer k that controls the number of candidates to be considered by the embedding approach, and a set of stys that can (optionally) be used for additional filtering. The algorithm internally uses a text annotator and a Knowledge Base ( [symbol] ) on which the annotator is also trained. In order to abstract from implementation details of different annotators, a general function is defined below.

Definition 1:

Function AnnotateText_{K} takes as input a text txt and returns a set of concepts {C₁, . . . , Cₙ} such that for every Cᵢ some substring str of txt exists such that str-sim(str, Cᵢ. [symbol] )≥thr, where str-sim is some similarity function and thr some threshold.

FIG. 5 is a flow chart showing a summary of the process. The process is shown in more detail in FIG. 6.

In FIG. 5, in step S1001, a semantic approach is used to determine from an ontology, entities that are linked to the query. If no entities are identified in step S1001, then the method proceeds to step S1003 where the possible entities are identified using an embedding method. If candidates are found in step S1001 or in step S1003, then these are filtered using semantic types in the S205 and the candidate concepts are output in step S1007.

This process will now be described in more detail with reference to the flow chart of FIG. 6. Here, as an input, a user text is provided in step S101 and this is annotated with concepts from a KB as described above in S102. This results in a set of concepts (txtAnn) describing the user text S103.

Next in step S104, there is a first attempt at generating candidate concepts (CandCons), by taking the concepts returned by the annotator, determining their descendants in the KB, and selecting those that are also in the PGM (TargetCons).

If no candidates can be computed in S105, then the more “relaxed” embedding approach described above is employed S106. Here, a list of concepts (ConsWithWeight) is extracted from the input text by selecting the concepts from the PGM that have labels which are most similar to the user text.

Next, a set of semantic types S₁ of the concepts A from the txtAnn S107 is generated. This set S₁ is then used to filter CandCons to extract concepts that have a semantic type or are linked to a concept with a semantic type that is found within S₁ S108. The top k list of these candidates is then returned S109 as CandCons.

Finally, candidates (CandCons) computed by either method S109 can optionally be further filtered according to a set of stys of interest S110.

The semantic approach is used at first because this is expected to be more selective and with higher precision (fewer false positives).

After computing an initial list of candidates, the most relevant of those needs is to be selected and passed to the dialogue-system. In a naïve approach, the user can be presented with the full list of candidates and asked to choose (an approach that is actually followed in some commercial SCDSs). Unfortunately, this approach is not user-friendly and still users may find it hard to pick the correct entities if the difference between two candidates is not clear to them. Even worse, this approach cannot be implemented in spoken dialogue-systems where the candidates need to be read to the user. A way to group the candidates according to some properties and ask the user which value of that property is most closely related to the condition they report is needed.

Continuing the above example, as can be seen, many of the potentially relevant concepts are about some kind of “Rash” which is further specialised with either the body location where it manifests (“Abdomen,” “Arm,” etc.) or its appearance (“Circular,” “Bumpy”). It can be assumed that these differences are also explicated in a medical KB using appropriate triples like the following ones:
<RashInAbdomen location Abdomen> <RashInArm location Arm>
<CircularRash shape Circular> <BumpyRash shape Bumpy>

This is shown in FIG. 4, where it can be seen that there is a semantic type for each of the triples. The semantic types of the objects in these triples (e.g., BodyPart for Abdomen and Arm, and Shape for Circular and Bumpy) provide potential category grouping of the candidates and can be used to ask questions that can help prune the search space. For example, from the above set of candidates the questions that arise are “Where is your Rash?” and “What shape is your Rash?” Moreover, potential answers for the first questions are “Abdomen,” “Arm,” or “None of the above,” the last of which includes candidates CircularRash and BumpyRash that are not connected in the KB with any body part.

Based on the above, Algorithm 2 is provided which is defined in pseudocode as follows:

Algorithm 2 CandidateSearch [symbol] (CandCons, styList, n)

| | Input: A set of candidate concepts and stys styList form some KB [symbol] and a positive |
|---|---|
| | integer. |
| 1: | while \|CandCons\| ≥ n do |

| | |
|---|---|
| 2: | Create a map styToCandidates such that for every sty ∈ styList we have |

| | |
|---|---|
| 3: | styToCandidates.sty :={ [symbol] C, C′ [symbol] \| C ∈ CandCons, C′ ∈ C.p,sty ∈ μ(C′)} |

| | |
|---|---|
| | Let styₘ be some key in styToCandidates with the most values. |
| 4: | if \|styToCandidates.styₘ\| < n then break |
| 5: | ansCons := askUser(styToCandidates.styₘ, styₘ) |
| 6: | if ansCons == ∅ then |

| | |
|---|---|
| 7: | then CandCons := CandCons \ π₁styToCandidates.styₘ |

| | |
|---|---|
| 8: | else |

| | |
|---|---|
| 9: | CandCons := ansCons |

| | |
|---|---|
| 10: | end if |

| | |
|---|---|
| 11: | end while |
| 12: | if \|CandCons\| > 1 then |

| | |
|---|---|
| 13: | CandCons := askUser({ [symbol] C′, ⊥ [symbol] \| C ∈ CandCons}, null) |

| | |
|---|---|
| 14: | end if |
| 15: | return CandCons |

An embodiment of CandidateSearch is depicted in the flowchart of FIG. 7. CandidateSearch takes as input a set of candidate concepts S202 (CandCons) (possibly computed by GenerateCandidates), a set of stys (stylist) over which grouping is done, and a positive integer (n) which is used to control the grouping process.

The algorithm enters a loop S203 where for each of the concepts in the candidate sets S204, it identifies concepts C′ that are connected to the candidate concepts C and builds a pair of the form <C,C′> S205. This is where C E CandCons and C′ is a concept to which C points. These pairs are then grouped into the semantic type of the associated concept C′ and formed into a map S206 with keys of the semantic types and values of the pairs such that the maps have the form {sty, <C,C′>}. A pair is built because the label of C′ will be used as an answer value for the question that would be generated. Subsequently, the algorithm selects the group that contains the most candidates and asks a question related to the type of that group S207. Alternate strategies include selecting the group based on a preferred semantic type for a given semantic type of C.

Generating the question to be asked for the selected group as well as the potential answer values is done using function askUser, which is discussed in detail below. The possible values of the answers also include a “None of the above” answer in which case this function returns the empty set. If this is the answer S210, then all candidates in the presented group are removed S211.

The algorithm stops the grouping process and exits the whole loop when the set of candidates has dropped below a threshold n S203. In this case, the set is considered sufficiently small that the remaining candidates be presented (or read) to the user and the user can then select the most relevant candidate.

Following through with the above example, the algorithm would create the following two groups:
styToCandidates.BodyPart:={<RashInAbdomen, Abdomen>, <RashInArm, Arm>}
styToCandidates.Shape:={<CircularRash, Circular>, <BumpyRash, Bumpy>}

In an embodiment, algorithm 2 generates two types of questions, one that asks users to clarify the value of a specific property of the candidates (line 5) and one that simply prints all candidates and asks users to choose one of them (line 13).

The generation of fluent and natural questions is a non-trivial problem. A simple but effective template-based shallow generation approach is used. The two types of questions are generated by the askUser function, which takes as input a pair of concepts and a semantic type. The pseudocode of this function is presented below.

Algorithm 3 askUser(ConceptPairs, sty)

| | Input: A set of pairs of concepts and a semantic type (sty) |
|---|---|
| 1: | if sty == null then |

| | |
|---|---|
| 2: | printIn “Which of the following?” |
| 3: | for [symbol] C, — [symbol] ∈ ConceptPairs do |

| | |
|---|---|
| 4: | printIn “\t ” + C.lay |

| | |
|---|---|
| 5: | end for |
| 6: | print “\t None” |
| 7: | ans := read answer from console |
| 8: | return {C \| C.lay = ans} |

| | |
|---|---|
| 9: | else |

| | |
|---|---|
| 10: | printIn fetchQuery(sty) |
| 11: | for [symbol] C, C′ [symbol] ∈ ConceptPairs such that C′.lay hasn't been printed before do |

| | |
|---|---|
| 12: | printIn “\t ” + C′.lay |

| | |
|---|---|
| 13: | end for |
| 14: | printIn “\t None” |
| 15: | ans := read answer from console |
| 16: | return all C such that [symbol] C, C′ [symbol] ∈ ConceptPairs with C′.lay = ans |

| | |
|---|---|
| 17: | end if |

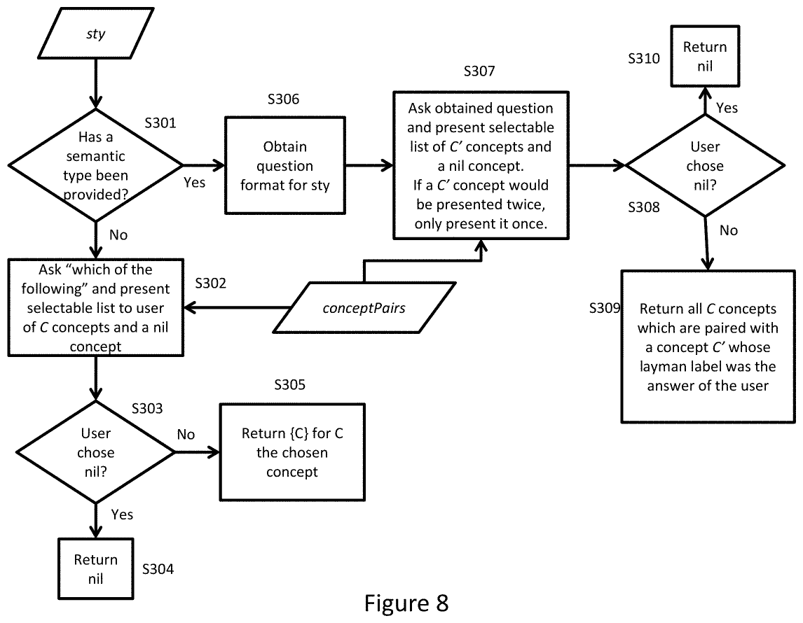

This function is also depicted on the flowchart in FIG. 8.

If the sty is null S301, then the algorithm proceeds with printing a question of the form “Which of the following?” and then prints the user-friendly label of each candidate S302. Since the set of candidates does not contain duplicates and by the assumption of uniqueness of user-friendly labels in the KB, these labels are unique. The user will then select a concept or the “None of the above” nil answer. If the answer is nil S303, then nil is returned by the function S304, otherwise the set {C} where C is the chosen concept is returned S305.

In case the semantic type provided is not null, then a specific query that depends on the semantic type needs to be rendered S306. A question has been assigned to each semantic type at design time. An excerpt of questions for a symptom-checking scenario is depicted in the following table:

| Semantic type | Question |
|---|---|
| BodyPart | “Where is the problem located?” |
| NeurologicalFinding | “Do you feel” |
| Severity | “How severe is your problem?” |
| BiologicalSubstance | “Do you see” |
| Appearance, Colour, | “Does it look:” |
| or Shape | |
| SpatialQualifier | “In which side?” |

As can be noted, these questions are quite general and neutral and fit most data and cases. The function will present this question to the user S307. Regarding answer values, the user-friendly label of the possible property value concepts are used. In this case, duplicates may exist. For example, the candidate selection step has returned with concepts C₁=HeadInjury and C₂=HeadPain which have been grouped according to body structure generating pairs <C₁, Head> and <C₂, Head>. In that case, the value answer for the question “Where is your symptom?” is the same for both concepts (“Head”). The algorithm takes care to print “Head” only once and if the user selects this, then both concepts C₁ and C₂ would need to be returned by the function S307. If the user chooses a labelled option S308, then all C concepts which are paired with the C′ that the user selected are returned by the function S309. Otherwise, if the user chose “None of the above” then the function returns nil.

As noted above, Algorithm 2 uses the properties of concepts in the KB in order to group the candidate concepts. It is clear that the more properties these concepts have and the more they share them with each other, the more effective these groupings would be. In a different case, groups will mostly contain a single concept and all the others would be in the “None of the above” answer. Medical KBs are known to be incomplete and underspecified.

For example, in SNOMED CT concept, RecentInjury is not associated with concepts Recent and Injury and SevereAbdominalPain is not linked with concept Severe. The same issue can easily be observed in other KBs like DBpedia, where the category ItalianRenaissancePainters is not connected to concepts Painter or ItalianRenaissance.

In an embodiment, to improve the effectiveness of the grouping strategy, all concepts in TargetCons need to be enriched with as many triples as possible. This task can be manual but this would be time-consuming and difficult to maintain. It has been noted that labels of concepts in (biomedical) ontologies is a good source of additional information. For instance, in the above example, it can be seen that the label of concept RashInHead implies a link between Rash and Head.

Building on this idea, a semi-automatic pipeline depicted in Algorithm 4 is used to extract such information from concept labels. The algorithm takes as input a set of concepts and uses their label to extract triples of the form [symbol] C p C′ [symbol] . To achieve this, in an embodiment, a text annotation service is used.

In step S401, for each concept C, further concepts C′ are extracted from the label of C by, for example, annotating the text as described above using “definition 1.”

To control the number of new triples extracted some list of stys of interest, (styList) can also be used. This can be implemented via step S403 that looks for extracted C's that are related to C via a semantic type, for example, location, etc.

Algorithm 4 conceptEnrichment [symbol] (TargetCons, styList, thr)

| | Input: A set of concepts and stys from some KB [symbol] and a real number thr |
|---|---|
| 1: | Inspect := ∅ |
| 2: | for all C ∈ TargetCons do |

| | |
|---|---|
| 3: | for all C′ ∈ AnnotateText [symbol] (C. [symbol] ) such that μ(C′) ∩ styList ≠ ∅ do |

| | |
|---|---|
| 4: | if score_{model}( [symbol] C C′ [symbol] ) ≥ thr then |

| | |
|---|---|
| 5: | Add [symbol] C p C′ [symbol] to [symbol] for some p with domain in μ(C) and range in μ(C′) |

| | |
|---|---|
| 6: | else |

| | |
|---|---|
| 7: | Inspect := Inspect ∪ { [symbol] C p C′ [symbol] } |

| | |
|---|---|
| 8: | end if |

| | |
|---|---|
| 9: | end for |

| | |
|---|---|
| 10: | end for |

Triples extracted from such an automated pipeline can be erroneous. In step S405, the extracted triples are evaluated. In one embodiment, these triples could be manually checked, however, this is almost equivalent to constructing the links manually. Thus, in an embodiment, techniques are used to score the extracted information and focus validation only on the low-scored pairs.

Several different methods can be used like KB embedding models, training a custom deep NN classifier using label embeddings, or training a traditional classifier using features like n-grams or the dependency-parse tree of concept labels. Some details of these are explained below.

If the extracted triple is valid, then it is added to an enhanced KB in step S407. The enhanced KB may be stored as part of the existing KB or stored separately.

The above methodology and framework is used in the system described with reference to FIG. 1 to build an interactive system that can be used to help understand vague user text in a symptom-checking dialogue system (SCDS).

For example, if a user enters text like “My stomach hurts” then subsequently the relevant nodes (symptoms) in a Probabilistic Graph Model (PGM) need to be activated to proceed with symptom-checking.

In the examples described below, a PGM is used that contains about 2261 symptoms which correspond to a small subset of a much larger medical Knowledge Base. In an embodiment, the medical KB can contain 1.5 m concepts, 173 properties, 1.8 million subsumption axioms, 2.2 m pref/alt labels, 93 semantic types, and 34 domain/range axioms.

In an example, 265 user text queries are collected from the above described SCDS and medical doctors were asked to map each of them to the most relevant concept in PGM; these concepts will be termed the user intended concepts. As another test, the input text queries were further modified by removing some of parts of the text in an attempt to make them more vague. For example, if the user text is “I feel a pain around heart” the modified version could be “I feel a pain.” To do this, sentence embeddings were used between original input text and labels of PGM concepts that appear in the object position of triples. For example, the triple <Pain findingSite Heart> and the label Heart. [symbol] =“Heart” were used to remove the respective text from the above sentence.

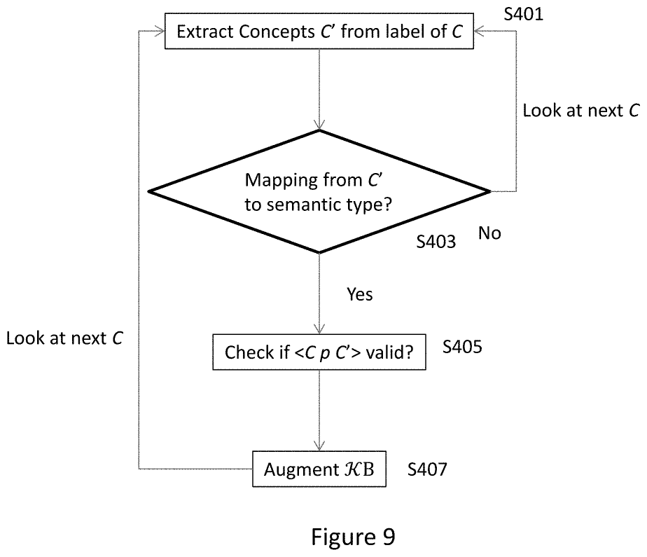

Algorithm 4 and FIG. 9 above discuss a concept enrichment process. This was used to extract additional triples for all PGM concepts. To control the process, a list of 10 stys of interest (parameter styList in Algorithm 4) was set. All extracted triples were scored (S405) using two different models and evaluated using 240 labelled data.

In the first method, the RESCAL (Nickel, M., Murphy, K., Tresp, V., Gabrilovich, E.: A review of relational machine learning for Knowledge Bases, Proceedings of the IEEE 104(1), 11-33 (2016)), approach was used for KB embeddings and this model yielded an AUC of 0.52 on the testing data.

The second approach was a three layer Neural Network with two hidden layers of sizes 3 and 5. The input was the concatenation of the sentence embeddings of the PGM node text and the extracted triple. Both embeddings were of size 512, yielding a combined input layer of size 1024. The training and test sets were of sizes 192 and 48, respectively, with the network trained using a binary cross-entropy loss function. Resulting accuracy was 0.84 while AUC was 0.73. This result is interesting in the sense that even simple custom approaches work better than off-the-shelf involved KB embedding approaches.

The enrichment process increased the triples of the 2261 symptoms from 3920 to 7102. The breakdown of these triples per sty before and after enrichment is depicted in Table 2.

TABLE 2
Counts of PGM concepts with the given semantic types
(stys) shown before and after concept enrichment.

| sty | Before | After | sty | Before | After |
|---|---|---|---|---|---|
| BodyPart | 1585 | 2566 | ClinicalFinding | 1049 | 1567 |
| ObservableEntity | 728 | 1102 | AbnormalBodyPart | 452 | 712 |
| QualifierValue | 31 | 652 | AnatomyQualifier | 42 | 218 |
| Substance | 17 | 139 | SpatialQualifier | 3 | 119 |
| Organism | 3 | 16 | ClinicalQualifier | 2 | 11 |

Possible stys body part, observable entity, abnormal body part, substance, organism, qualifier value, clinical finding, anatomy qualifier, spatial qualifier and time patterns, and time duration.

From these numbers it can be concluded that, in spite of the enrichment, some stys will most likely not be very effective in grouping candidates, as they do not appear often in the PGM nodes (e.g., all below a count of 100). Below it will be investigated further which of those stys were actually the most frequently used ones when we run the main algorithm on input user text.

Next, the effect of evaluating k in selecting the top-k candidates, specifically determining the number of top-k candidates that are needed to compute in order for this set to always include the user intended concept. To evaluate the sensitivity of the embedding approach on the selection of k, the value of k was varied starting with k=5, and using increments of 5 it was established in how many cases (out of the 265) the intended user concept was in the top-k candidates. The results are presented in Table 3.

TABLE 3
Number of additional correct concepts included in the candidates
after increasing k by 5, as well as for above 30.

| k value | 5 | 10 | 15 | 20 | 25 | 30 | >30 |
|---|---|---|---|---|---|---|---|
| Degenerated | 91 | 26 | 20 | 14 | 10 | 8 | 96 (36%) |
| Full Queries | 229 | 16 | 5 | 4 | 1 | 2 | 8 (3%) |

As can be seen for k=30, the embedder was able to include the user intended concept in 257 (97%) and 169 (64%) cases in two different test query sets in contrast to 245 (92%) and 117 (44%) for k=10, which is the usual one. As can be seen for vague (modified) queries going above top-10 is highly beneficial.

Next, candidate selection and depth of correct answer was evaluated. Here, two variations of the candidate selection algorithm (Algorithm 1) were evaluated. The first implements Algorithm 1 as presented, that is, it first uses subClassOf traversal on the KB (line 2 of Algorithm 1) and if this step returns an empty set, then it falls back to embeddings, while the second only uses the embedding approach. In both cases k was set to 30 for the embedder.

Table 4 shows the number of times that the correct answer was included in the candidate set computed by the two algorithms in the two different sets of queries.

TABLE 4
Frequency where correct answer was returned by algorithms
(correct cases/cases that approach was applied).

| | KB + Embedder | | |
|---|---|---|---|
| Input Text | KB | Embedder | Embedder (only) |
| Degenerated | 90/197 | 38/68 | 169/265 |
| Full Queries | 181/247 | 15/18 | 257/265 |

The first approach was further broken down and it was measured in how many cases the KB descendant approach returned a non-empty set of candidates, and in how many of them the intended concept was within the candidates. As can be seen, there are quite a few cases where the KB approach returns an empty set of candidates (68 in the first and 18 in the second set of queries) and the fallback needs to be employed. Moreover, even in the cases when the KB approach returns a non-empty set, in quite a few of them, this set does not contain the intended user query. This is because the current implementation of checking descendants in the KB is semantically “very strict” compared to the flexibility of a query that can be formed in text. For example, the behaviour of this approach is considerably different if the input text is “Blood in stool” or “Bleeding.”

In this case, the annotator associates KB concepts of even different sty. In contrast, the embedder works considerably better due to the fact of loosely capturing the semantics and similarities between concepts.

After candidate's selection, we want to evaluate how many questions would be required before Algorithm 2 returned the user intended concept. This is similar to the number of clicks (scan effort) in faceted search evaluation. Only the cases where the candidate selection approach did manage to return the user intended concept in the set of candidates are considered. The results are depicted in Table 5; the format X (Y) means that in X number of cases the intended user concept is reachable after Y questions. As can be seen, most concepts are reachable after one or two questions and all concepts are reachable at most after four.

TABLE 5
Number of cases and questions required to reach the intended concept.

| | KB + Embedder | | |
|---|---|---|---|
| Input Text | KB | Embedder | Embedder (only) |
| Degenerate | 48(1), 31(2), 8(3), 3(4) | 10(1), 25(2), 3(3) | 68(1), 67(2), 32(3), 2(4) |
| Full Queries | 115(1), 53(2), 12(3), 1(4) | 3(1), 10(2), 2(3) | 50(1), 127(2), 71(3), 9(4) |

TABLE 6
Left: stys that appeared in evaluation (% of total tests);
Right: Number of answers per question (median; mean)

| | |
|---|---|
| Anatomical structure | 75% |
| Qualifier value | 36% |
| Observable entity | 11% |
| Morphologically altered Str. | 6.3% |

| | KB + Embedder | | |
|---|---|---|---|
| Input Text | KB | Embedder | Embedder |
| Degenerated | 4; 26.2 | 15; 12.3 | 13; 11.8 |
| Full Queries | 4; 4.3 | 13; 11.0 | 6; 9.8 |

Next, the grouping algorithm was further evaluated. Table 6 (left) shows which stys were used to group the set of candidates in any of the user queries of our test sets. Results were very similar in all variations run. In 51% of tests, the final question of the algorithm did not have a sty to distinguish the correct answer from the others (although previous questions would have), so the answers were collapsed to a generic question. Ideally the groups created by the algorithm should neither be very small (which leads to too many questions) nor too large (which leads to few questions with too many answers).

Summary statistics for these results are shown in Table 6 (right). It can be seen that the modified text queries result in larger answer sets since they are more vague. One other aspect to note is the small answer sets with the annotation filter than with the embedding filter. This comes as a trade off against the lower recall of the annotation filter, which we can interpret as a more homogeneous set of candidates being returned by the stricter filter.

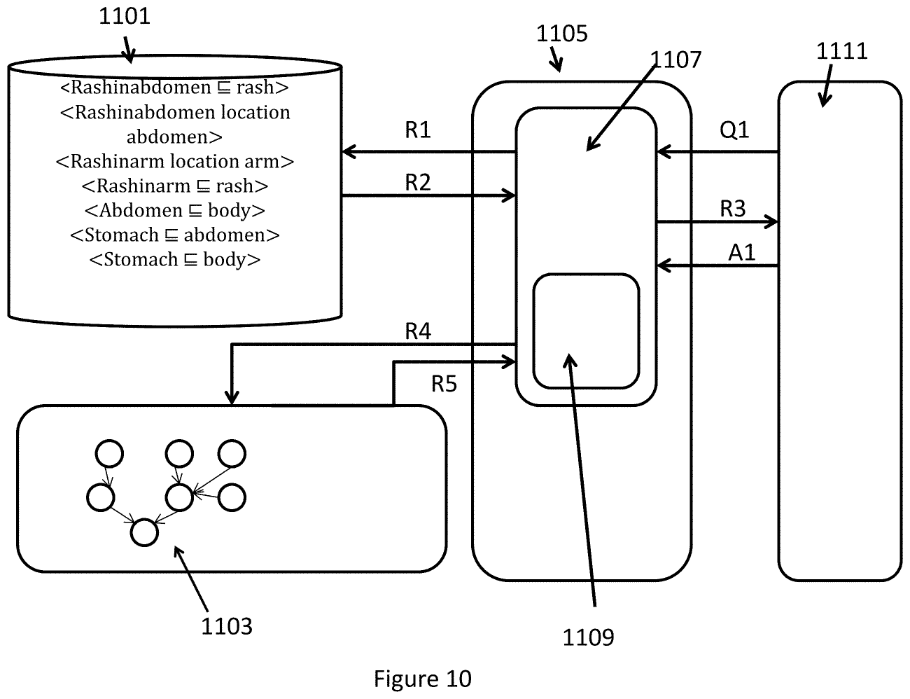

As a summary, FIG. 10 shows a system in accordance with an embodiment. The system comprises a database 1101, the database can be any type of mass storage for hard disk drives, RAID systems, solid state drives, holographic memory, and removable storage, such as USB. In an embodiment, the database stores an ontology, for example, a medical knowledge base (KB). In the ontology, the data is stored in the form of triples where the relationship between concepts is stored, for example, the concept Rashinabdomen is stored as <Rashinabdomen⊆rash> to encode that Rashinabdomen is within the concept rash. Also, in this example, it is stored as the triple <Rashinabdomen location abdomen> where “location” is a property. The sty for RashInAbdomen is ClinicalFinding and the sty for Abdomen is BodyPart. This information is encoded using a different set of triples:
<RashInAbdomen has_sty ClinicalFinding>
<Abdomen has_sty BodyPart>

In this embodiment, the ontology is stored in the database 1101 with forward chaining where all relationships derivable in the ontology are stored as triples; for example, if the ontology has the triples, <Abdomen⊆body> and <Stomach⊆abdomen>, then through forward chaining also saves the triple <Stomach⊆body>.

A second mass storage 1103 is provided that stores the inference engine and PGM. The PGM contains a plurality of nodes and the probabilistic relationships between those nodes. Some of the nodes of the PGM relate to diseases and others to symptoms. Once a node relating to a symptom has been selected (activated), the possible diseases/causes related to that symptom can be identified and then questions can be constructed to narrow down the possible causes/diseases using known probabilistic inference techniques such as importance sampling. Further, value of information measures can be used to determine the most suitable further questions. How such further questions are established is outside the scope of this application and here, the embodiment is to activate the single most appropriate node in the PGM. To do this, the ontology of the first mass storage 1101 is aware of the nodes that link directly to nodes in the PGM.

It should be noted that the first 1101 and second 1103 mass storage can be separate or a combined storage.

The first mass storage is in communication with a server 1105. The server comprises a processor 1107 that runs program 1109. The server 1105 is in communication with user terminal 1111 that may be a mobile phone or the like. In an embodiment, the user terminal 1111 receives user query Q1 over a mobile telephone network or the like. The processor 1107 running program 1109 divides the user inputted query into words and communicates with database 1101 by sending requests R1 and receiving responses R2 to retrieve candidate concepts as described in relation to algorithm 1 and FIGS. 5 and 6.

The program then takes the triples retrieved from the database 1101 and using their semantic types, which are defined in the triples, groups them and performs the methods of FIGS. 7 and 8 to output a question to the user R3. The user then sends a response A1, if this is selecting an answer that corresponds to a specific node of the PGM and a request R4 is sent to the inference engine to activate the corresponding node and then the inference engine controls the remainder of the diagnosis R5.

It should be noted that in the above, the information that needs to travel either from the user's terminal to the server or between the server and the database is kept low.

The above embodiments relate to the problem of interpreting and understanding vague and imprecise user queries using KBs. This problem is highly relevant in applications like dialogue-systems and virtual assistants where the input query needs to be mapped to some entity that activates a background service.

The above methods allow a symptom-checking dialogue system to operate where users can enter text like “I am not feeling well,” “I sleep terribly,” and more, which have a mismatch compared to the entities that are usually found in formal medical ontologies. The above embodiments bridge the gap between user queries and a set of pre-defined (target) ontology concepts. The above embodiments show how the ontology and statistical techniques can be used to select an initial small set of candidate concepts from the target ones and how these can then be grouped into categories using their properties in the ontology. Using these groups, the user questions can be configured in order to try and reduce the set of candidates to eventually a single concept that captures the initial user intention.

To further improve the effectiveness of this approach, in an embodiment, a concept enrichment pre-processing step is provided based on information extraction techniques.

In all previous works on dialogue-systems, it is assumed that users report their requests in a clear and precise way and the relevant information is extracted using machine learning-based slot-filling techniques. Unfortunately, in many cases, user requests are highly imprecise, incomplete, and vague and this is particularly the case in complex applications like symptom-checking. In such a case, no single frame or pre-defined slots can be identified and different symptoms may exhibit great heterogeneity in their structure and properties. Due to this complexity of the medical domain, all previous approaches simplify the problem by handling only a specific sub-domain of medicine and support only a small set of symptoms (at most 150). However, commercial symptom-checking systems that attempt to support primary care include many hundreds of symptoms. To tackle this problem, a framework has been developed which can construct on the fly, a small dialogue that asks the user a few “clarification” questions in an attempt to “activate” the proper entity from the KB. An initial (small) set of candidates is produced using semantic and ML-based techniques and then the properties of these candidates in the KB are used to group them. The “most relevant” group is selected and one question is printed. To improve the effectiveness of the approach and overcome underspecification issues of KBs, an enrichment information extraction pipeline was designed and various scoring models were used to assess the soundness of the extracted information.

The above embodiments combine, extend, and adapt in a non-trivial way ideas from dialogue-systems as well as guided (faceted) navigation and extends previous approaches to mapping keywords to ontologies. The above embodiments build in a dynamic way a mini-dialogue with the purposes of understanding vague user queries and which uses KBs to such an extent. To do so, many scientific and engineering challenges had to be addressed like enriching the KB concepts, determining the set of stys, designing a grouping algorithm, and performing analysis to determine its effectiveness. The above embodiments also provide a first insight on building such a dynamic system analysing the number of questions and answer set size as well as the sensitivity of k in top-k candidate selection. Although the above embodiments mainly relate to symptom-checking, they are relevant and useful in any domain and can greatly contribute towards building more user-friendly and intelligent systems.

Whilst certain embodiments have been described, these embodiments have been presented by way of example only, and are not intended to limit the scope of the inventions. Indeed, the novel devices, and methods described herein may be embodied in a variety of other forms; furthermore, various omissions, substitutions, and changes in the form of the devices, methods, and products described herein may be made without departing from the spirit of the inventions. The accompanying claims and their equivalents are intended to cover such forms or modifications as would fall within the scope and spirit of the inventions.

### APPENDIX A

### Definitions

In this specification, the term “Simple Concept” means an elementary entity intended to refer to some real-world notion and is interpreted as a set of things. Examples of simple concepts are: Human, Male, TallPerson, and Chairs. Simple concepts are also referred to as “Atomic Concepts” and in OWL jargon, concepts are also called “Classes.”

A “Role,” “Relation,” or “Property” is an entity that denotes relations between objects. Examples of this are hasChild, hasDiagnosis, and isTreatedBy.

The symbol Π represents logical conjunction. It is called AND for short. It can be used to form the conjunction of two concepts and create a new one. The conjunction of two concepts is interpreted as the intersection of the sets to which the two concepts are interpreted. For example: Professor Π Male, which represents the notion of a male professor. As a whole, it is a concept. It is interpreted as the intersection of the sets to which concepts Professor and Male are interpreted.

The symbol ∃ (a reversed capital letter E) is defined as the existential operator. It is called EXISTS for short. It can be used with a role and possibly combined also with a concept to form a new concept. For example: ∃hasChild represents the set of all things that have some child. Also: ∃hasChild.Male represents the set of all things that have a child, where the child is male.

The symbol [symbol] means “entails.” It is used to denote that something follows logically (using deductive reasoning) from something else. For example: ∃hasChild.Male [symbol] ∃hasChild since if someone has a child which is male, then it follows that they necessarily have some child.

The symbol ⊆ is defined as the subclass operator (or the inclusion operator). It denotes a subclass relationship between two concepts. If one concept C is a subclass of another concept D, then the set to which C is interpreted must be a subset of the set to which D is interpreted. It can be used to form axioms. Intuitively it can be read as IF-THEN. For example: Male⊆Person can be read as “If something is a male then it is also a person.”

The symbol ⊆ has the standard set theoretic meaning of a subset relation between sets.

The difference between the symbol ⊆ and ⊆ is that the latter denotes inclusion relation between classes. Classes are abstractions of sets. They don't have a specific meaning, but meaning is assigned to them via interpretations. So, when Male is written as a class, it acts as a placeholder for some set of objects. Hence Male ⊆ Person means that every set to which Male is interpreted is a subset of every set that Person is interpreted. This relation is written as:
Male^{J}⊆Person^{J}

Where J is called an interpretation and it is a function that maps classes to sets. Hence, Male^{J} is a specific set of objects.

An “axiom” is a statement or property about our world that must hold true in all interpretations. An axiom describes the intended meaning of the symbols (things). Male⊆Person is an example of an axiom.

A “knowledge base” or “ontology” is a set of axioms which describe our world. For example, the knowledge base {Male⊆Person, Father⊆hasChild.Person} contains two axioms about our world; the first is stating that every male is also a person (the set to which Male is interpreted is a subset of the set to which Person is interpreted), while the latter is stating that every father has a child that is a person (the set to which Father is interpreted is a subset to the set of things that have a child that is a Person). There are several well-known publically available medical ontologies (e.g., UMLS, FMA, SNOMED, NCI and more).

A “complex concept” is an expression built using simple concepts and some of the aforementioned operators. The resulting expression is again a concept (an entity denoting some set of things). Professor H Male as used above is an example of this. A further example is Person Π ∃ hasChild.Male, where Person and ∃ hasChild.Male are two concepts and Person Π ∃ hasChild.Male is their conjunction. This complex concept is interpreted as the intersection of the sets to which Person is interpreted and to which ∃ hasChild.Male is interpreted. Intuitively this expression intends to denote the set of things that are persons and have a child that is a male.

The term “concept” can refer to either simple concepts or complex concepts.

A knowledge base (KB) (or ontology) can entail things about our world depending on what axioms have been specified in it. The following example is provided to aid the understanding of this idea and the definitions above.

Let [symbol] be the following ontology:

[symbol] [symbol] {Female⊆Person, HappyFather⊆∃ hasChild.Female, ∃ hasChild.Person⊆Parent}.

Then from this, it can be deduced that:

[symbol] [symbol] HappyFather⊆∃ hasChild.Person

This inference can be made because given the ontology that every female is a person and a happy father must have at least one child that is a female, it follows using deductive reasoning that every happy father must have a child that is a person.

Similarly, it can also be inferred that [symbol] [symbol] HappyFather⊆Parent.

An “IRI” is an Internationalized Resource Identifier, which is a string of characters that identifies a resource. It is defined as a new internet standard to extend upon the existing URI uniform resource identifier, the commonly used URL is a type of URI relating to location.

A “reasoning algorithm” (or reasoning system) is a mechanical procedure (software program), which given an ontology (aka knowledge base) can be used to check the entailment of axioms with respect to the knowledge specified in the ontology. In the previous example, [symbol] can be loaded to some reasoning algorithm and then check if HappyFather Parent is entailed by [symbol] . Reasoning algorithms are internally based on a set of “inference rules” which they apply iteratively on the axioms of the ontology and the user query in order to determine whether the axiom is entailed or not. Depending on the set of inferences rules that a reasoning system implements, it may or may not be able to discover the entailment of an axiom even in cases that this is actually entailed. A reasoning system may implement a “weak” set of inference rules in order to be able to handle large ontologies in a scalable way, whereas other reasoning systems may favour to answer correctly all cases and hence implement a more expressive set of inference rules. The former usually implements only deterministic inference rules, whereas the latter a non-deterministic one.

A “triple-store” is a particular type of reasoning system that supports entailment of ontologies expressed in the RDF(S) standard. Such reasoning systems are generally efficient and scalable, however, if the ontology is expressed in more expressive standards like OWL, they will not be able to identify all entailments. For example, standard triple-stores will not be able to answer positive on the query HappyFather Parent over the ontology [symbol] in the previous example.

For a set of tuples of the form tup={<k₁,v₁>, <k₂,v₂>, . . . , <kₙ,vₙ>}, πᵢtup where i∈{1, 2} is called the projection of tup on the first or second argument and returns the set {k₁,k₂, . . . , kₙ} or {v₁,v₂, . . . , vₙ}.

A map is a collection of key/value pairs. Maps can be semi-structured, meaning that values associated with different keys may be of a different type. For Map, a map and k, some key the notation Map.k is used to denote the value associated with k. If no value exists for some key k, then Map.k:=v, which means that a new key is added to the map and its value is set to v.

1. A method for reducing the number of potential matches of entries in a database to a user inputted query, the method comprising:
receiving a user inputted query;
identifying a plurality of candidate entries in said database that provide a match to said user inputted query, wherein the entries in the database are concepts in a medical knowledge base and are stored in the form of triples, said triples comprising a first concept, a second concept and a relation between the first concept and the second concept, wherein the relation is selected from a plurality of relations, one of which is semantic type, the semantic type being selected from: body part, observable entity, abnormal body part, substance, organism, qualifier value, clinical finding, anatomy qualifier, spatial qualifier and time patterns, time duration;
grouping the plurality of candidate entries on the basis of their associated semantic type derived from the relation in the medical knowledge base;
selecting the group with the largest number of entries; and
transmitting a request to a user to select between the entries in the group with the largest number of entries with the same semantic type.

2. (canceled)

3. A method according to claim 1, wherein a subset of the concepts in the medical knowledge base are target concepts, wherein said method is adapted to provide matches to said target concepts.

4. A method according to claim 3, wherein said target concepts correspond to nodes in a probabilistic graphical model.

5. (canceled)

6. A method according to claim 1, wherein the transmitted request additionally comprises a request based on candidate entries other than from those in the selected group.

7. A method according to claim 1, wherein the user is asked to select between the group with the largest number of entries if the largest number of entries is in excess of a threshold.

8. A method according to claim 1, wherein identifying a plurality of candidates comprises determining nearest neighbours from said database entries when mapped to the same embedded space as the query.

9. A method according to claim 1, wherein identifying a plurality of candidates comprises looking for a semantic match between entries in the database and said query.

10. A method according to claim 3, wherein said matches to target concepts are determined by:
annotating the query by selecting concepts from the medical knowledge base that have a label that is similar to the query;
determining matches to target concepts from the selected concepts by determining from the medical knowledge base all concepts descended from the selected concepts and keeping only those that are also target concepts.

11. A method according to claim 3, wherein said matches to target concepts are determined by:
annotating the query by selecting concepts from the medical knowledge base that have a label that is similar to the query to obtain first selected concepts;
identifying the semantic types of these first selected concepts;
annotating the query by selecting concepts from the target concepts that have a label that is similar to the query to obtain second selected concepts;
identifying the semantic types of these second selected concepts; and
determining matches to said target concepts from second selected concepts that have a semantic type that matches with one of the semantic types of the first selected concepts.

12. A method according to claim 3,
wherein said matches to target concepts are determined by a first process and a reserve process,
wherein said reserve process is used if the first process does not produce any matches, said first process comprising:
annotating the query by selecting concepts from the medical knowledge base that have a label that is similar to the query;
determining matches to target concepts from the selected concepts by determining from the medical knowledge base all concepts descended from the selected concepts and keeping only those that are also target concepts,
said reserve process comprising:
annotating the query by selecting concepts from the medical knowledge base that have a label that is similar to the query to obtain first selected concepts;
identifying the semantic types of these first selected concepts;
annotating the query by selecting concepts from the target concepts that have a label that is similar to the query to obtain second selected concepts;
identifying the semantic types of these second selected concepts; and
determining matches to said target concepts from second selected concepts that have a semantic type that matches with one of the semantic types of the first selected concepts.

13. A method according to claim 1, further comprising a method of pre-processing the database prior to identifying a plurality of candidate entries, wherein the pre-processing comprises producing a triple for indirectly related concepts which are related through multiple directly related concepts.

14. A method according to claim 1, further comprising a method of pre-processing the database prior to identifying a plurality of candidate entries, each concept in the database having a label, the method of pre-processing comprising:
identifying secondary concepts from the label;
determining a relationship from the label between a secondary concept identified in the label and the concept; and
saving the concept, secondary concept, and relationship as a triple.

15. A method of pre-processing a database,
wherein the entries in the database are concepts in a medical knowledge base and are stored in the form of triples, said triples comprising a first concept, a second concept, and a relation between the first concept and the second concept,
wherein the relation is selected from a plurality of relations, one of which is semantic type, the semantic type being selected from: body part, observable entity, abnormal body part, substance, organism, qualifier value, clinical finding, anatomy qualifier, spatial qualifier and time patterns, time duration, each concept in the database having a label, the method comprising:
identifying secondary concepts from the label of a concept;
determining a relationship from the label between a secondary concept identified in the label and the concept, the relationship comprising a category of interest in the medical knowledge base; and
saving the concept, secondary concept, and relationship as a triple.

16. (canceled)

17. A system for reducing the number of potential matches of entries in a database to a user inputted query, the system comprising:
an input adapted to receive a user inputted query;
a processor adapted to:
identify a plurality of candidate entries in said database that provide a match to said user inputted query, wherein the entries in the database are concepts in a medical knowledge base and are stored in the form of triples, said triples comprising a first concept, a second concept and a relation between the first concept and the second concept, wherein the relation is selected from a plurality of relations, one of which is semantic type, the semantic type being selected from: body part, observable entity, abnormal body part, substance, organism, qualifier value, clinical finding, anatomy qualifier, spatial qualifier and time patterns, time duration;
group the plurality of candidate entries on the basis of their associated semantic type derived from the relation in the medical knowledge base; and
select the group with the largest number of entries; and
an output for transmitting a request to a user to select between the entries in the group with the largest number of entries, with the same semantic type.

18. A system according to claim 17, wherein the input comprises a text input adapted to receive typed inputted text or a voice input.

19. (canceled)

20. A system according to claim 17, further comprising an inference engine, the inference engine having a probabilistic graphical model, wherein a subset of the concepts in the medical knowledge base are target concepts, wherein said method is adapted to provide matches to said target concepts, the target concepts corresponding to nodes in said probabilistic graphical model.
